(19) United States
(12) Patent Application Publication
(10) Pub. No.: US 2016/0018307 A1
(43) Pub. Date: Jan. 21, 2016

(54) Sample Extraction Apparatus And Method For Liquefaction Assessment

(71) Applicant: Hokoku Engineering Co., Ltd., Osaka (JP)

(72) Inventor: Chol-Ho Kim, Osaka (JP)

(73) Assignee: Hokoku Engineering Co., Ltd., Osaka (JP)

(21) Appl. No.: 14/871,804

(22) Filed: Sep. 30, 2015

Related U.S. Application Data
(63) Continuation of application No. PCT/JP2013/002295, filed on Apr. 2, 2013.

(51) Int. Cl. G01N 3/32; G01N 1/04; G01N 33/24

(52) U.S. Cl. CPC G01N 3/32; G01N 33/24; G01N 1/04; G01N 2203/0284

(57) ABSTRACT

A sample extraction apparatus for extracting samples from a target ground in order to assess whether liquefaction occurs in the target ground, includes: a casing; a holding sleeve, installed inside the casing, for holding the samples; and a vibrator configured to apply vibration accelerations corresponding to different seismic intensities to the samples in the casing, respectively, after the casing is inserted into the target ground to hold the samples of the target ground in the holding sleeve, so that a different liquefaction condition is provided for each sample, whereby said apparatus is configured such that, after having applied different vibration accelerations to the samples in the casing inserted into the target ground, respectively, said apparatus can resultantly extract the samples so that a liquefaction assessment can be made under a plurality of seismic conditions.

### CROSS REFERENCE TO RELATED APPLICATIONS

This is a continuation application of International Patent Application No. PCT/JP2013/002295 filed on Apr. 2, 2013 of which full contents are incorporated herein by reference.

### BACKGROUND OF THE INVENTION

1. Field of the Invention

The present invention relates to a sample extraction apparatus and a sample extraction method, for the purpose of making a liquefaction assessment of a ground.

2. Description of the Background Art

Many technologies have been developed as measures against liquefaction. In order to take such measures, there has been a need to initially predict liquefaction of on-the-spot soil.

The liquefaction of the soil has been assessed on the basis of e.g. the guidelines in “Recommendations for Design of Building Foundations (2001 edition)” authored by the Architectural Institute of Japan.

The liquefaction assessment has required various sorts of soil information. For this reason, various values of test results have been obtained for calculation such as: an N-value and soil properties to be obtained by a standard penetration test (boring); an FC-value to be obtained by a soil test; a u-value, a VS-value, a VP-value to be obtained by a sample extraction apparatus and method for the liquefaction assessment.

On the basis of the various sorts of information thus obtained, the calculation has been performed through the use of an FL method of e.g. Specifications for Highway Bridges (2002), and also through the use of a PL method provided by Iwasaki et al. (1980).

As another method, there has also been well known a liquefaction assessment method disclosed in Patent Document 1.

In such an assessment method, initially, a vertical steady excitation has been conducted at an excitation point set on a surface of a target ground to generate Rayleigh waves. Subsequently, a vertical amplitude of the Rayleigh waves and a passing time “Δt” thereof have been measured at a measuring point for each frequency. Subsequently, a measured passing time “Δt” has been used to calculate a phase velocity “c” of the Rayleigh waves for each frequency so as to obtain dispersion characteristics, and an S-wave velocity structure of the target ground has been determined on the basis of a dispersion curve representing the dispersion characteristics. Subsequently, the S-wave velocity structure has been used to specify a target layer, and the phase velocity “c” and the corresponding frequency “f” have been calculated in the target layer, and the vertical amplitude “w” and the phase velocity “c” of the Rayleigh waves corresponding to the frequency “f” have been applied to a predetermined calculation formula so as to calculate an internal attenuation “h” of the target layer. Subsequently, the calculated internal attenuation “h” has been used to estimate properties of a soil of the target layer, and the need for a liquefaction assessment has been determined.

### PRIOR ART DOCUMENTS

### Patent Documents

### Patent Document 1: Japanese Patent Application Publication No. 2002-55090

### PROBLEM TO BE SOLVED

In the conventionally conducted liquefaction assessment method described above, the following problems have occurred:

[1] In order to obtain the above-described values for calculation for the assessment, there has been a need to obtain the test results such as: the N-value and the soil properties obtained by the standard penetration test (boring); the FC-value obtained by the soil test; the u-value, the VS-value, the VP-value obtained by the sample extraction apparatus and method for the liquefaction assessment.

[2] Such values and formula have been obtained as a result of the accumulation of previous experiments in which setting up many uncertain hypothetical conditions has been needed. Due to such hypotheses, there occurred a problem that many areas, which should not be liquefied according to results of conventional assessments made in Urayasu City in Chiba Prefecture, were liquefied during e.g. the Great East Japan Earthquake.

[3] Even with calculation results based upon a larger number of hypothetical conditions, further hypotheses have been needed in determining how to apply the values of such calculation results to the liquefaction assessment.

[4] There has been another method of assessment based upon the experiment using a model; however, the method has needed again various hypothetical conditions and expensive equipment.

[5] The method disclosed in Patent Document 1 as shown in FIG. 7 has also depended upon various hypothetical conditions and calculations in such a manner that: a measured passing time “Δt” has been used to calculate a phase velocity “c” of the Rayleigh waves for each frequency so as to obtain dispersion characteristics, and an S-wave velocity structure of the target ground is determined from a dispersion curve representing the dispersion characteristics; and the S-wave velocity structure has been used to specify a target layer, and the phase velocity “c” and the corresponding frequency “f” have been calculated in the target layer and the vertical amplitude “w” and phase velocity “c” of the Rayleigh waves corresponding to the frequency “f” have been applied to a predetermined calculation formula so as to calculate the internal attenuation “h” of the target layer; and the calculated internal attenuation “h” has been used to estimate properties of a soil of the target layer, and the need for a liquefaction assessment has been determined.

### SUMMARY OF THE INVENTION

### Means for Solving Problems

In order to solve the above-described problems, there is provided, as one aspect of the present invention, a sample extraction apparatus for extracting samples from a target ground in order to assess whether liquefaction occurs in the target ground, including: a casing; a holding sleeve, installed inside the casing, for holding the samples; and a vibrator configured to apply vibration accelerations corresponding to different seismic intensities to the samples in the casing, respectively, after the casing is inserted into the target ground to hold the samples of the target ground in the holding sleeve, so that a different liquefaction condition is provided for each sample, whereby said apparatus is configured such that, after having applied different vibration accelerations to the samples in the casing inserted into the target ground, respectively, said apparatus can resultantly extract the samples so that a liquefaction assessment can be made under a plurality of seismic conditions.

Furthermore, there is provided, as another aspect of the present invention, a sample extraction method for the purpose of assessing as to whether liquefaction occurs in the target ground, including the steps of: inserting the casing into the target ground to hold the samples of the target ground in the holding sleeve, by using the above sample extraction apparatus; thereafter, applying vibration accelerations corresponding to different seismic intensities to the samples in the casing so as to provide the samples with different liquefaction conditions, respectively, by using the above sample extraction apparatus; and thereafter, retrieving aboveground each sample, extracted by using the above sample extraction apparatus, for making a liquefaction assessment.

### Advantageous Effects

According to the sample extraction apparatus and method for the liquefaction assessment as one of the present inventions described above, there can be achieved the following advantageous effects:

[1] The apparatus and method according to the present invention are based upon technical ideas of providing the sample inside the casing inserted into the ground with liquefying conditions on purpose so as to liquefy the sample inside the casing.

[2] Since such a sample liquefied proactively as described above has a squashily-liquefied range clearly different from a compact and sound layer without any liquefaction, anybody can assess the range by sight or touch.

[3] As described above, it is possible to assess a depth and range of the liquefied layer not by the calculations based upon the hypothetical conditions but by the actually extracted on-the-spot soil, thereby eliminating the need for various soil tests and the calculations based upon the hypothetical conditions while enabling the liquefaction assessment for the actually extracted soil within a short period of time.

[4] It is possible to show the actually liquefied soil to clients placing an order for buildings or various structures so as to make explanations and persuade the clients without any calculation results based upon the hypothetical conditions, thereby securing a high degree of reliability of the clients.

### BRIEF DESCRIPTION OF THE DRAWINGS

For more thorough understanding of the present invention and advantages thereof, the following descriptions should be read in conjunction with the accompanying drawings in which:

FIG. 1 depicts an explanatory view showing a step of inserting into a ground a sample extraction apparatus for a liquefaction assessment as an embodiment according to the present invention.

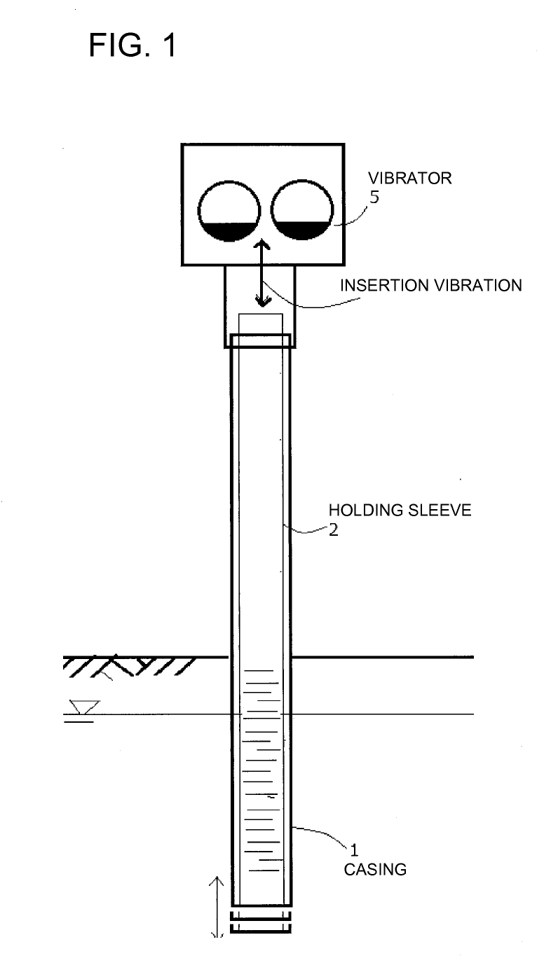

FIG. 2 depicts an explanatory view showing a step of promoting the liquefaction after the insertion of the sample extraction apparatus.

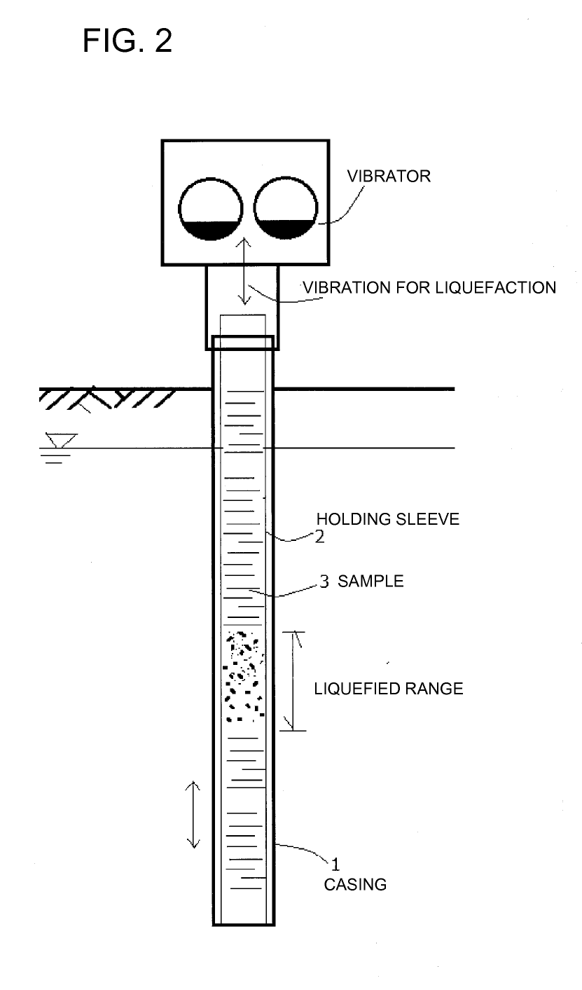

FIG. 3 depicts an explanatory view showing another structure for applying vibration accelerations to the sample.

FIG. 4 depicts an explanatory view showing still another structure for applying vibration accelerations to the sample.

FIG. 5 depicts a view showing an example of the extracted sample.

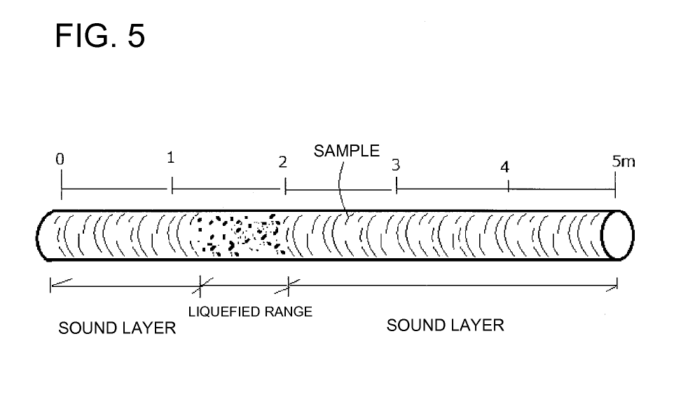

FIG. 6 depicts a view showing a relationship between seismic intensities and vibration accelerations.

FIG. 7 depicts an explanatory view of a method disclosed in Patent Document 1.

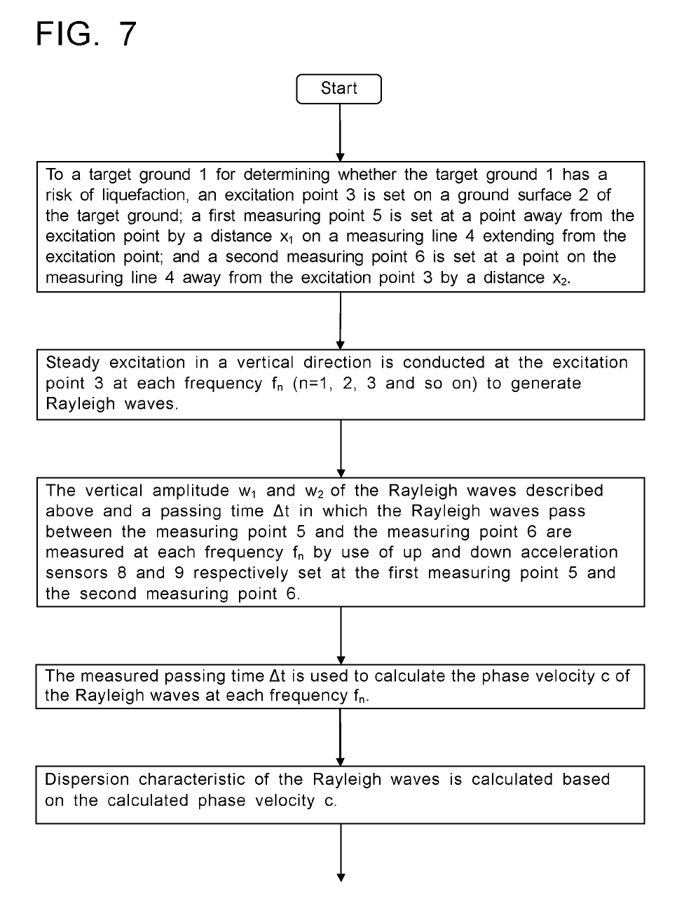

### DESCRIPTION OF EMBODIMENTS OF THE INVENTION

Hereinafter, a preferred embodiment of a sample extraction apparatus for a liquefaction access will be described in detail below with reference to accompanying drawings.

### EXAMPLES

### [1] Basic Concept of the Invention

As an embodiment according to the present invention, there are provided apparatus and method by which the actually liquefied on-the-spot soils applied with vibration accelerations corresponding to seismic intensities can be extracted, without any calculations based upon hypotheses, as samples of liquefaction conditions along a depth direction.

Since a liquefied range of the sample thus recovered can be assessed by sight or touch, anybody can recognize the depth and range.

### [2] Extraction Apparatus

The sample extraction apparatus includes: a casing 1; a holding sleeve 2 installed inside the casing 1 for holding samples; and a vibrator 5.

The casing 1 is configured such that the casing 1 is applied with vibration, rotation, or both, and is inserted along with the holding sleeve 2 into the ground.

Various sorts of sampling may also be carried out in accordance with a sort and conditions of a soil, such as thin-walled sampling, Denison (double pipe) sampling, core pack sampling, triple pipe sampling, special sand sampler for a soft/loose sandy soil.

The thin-walled sampling is applied to a sandy soil containing a large amount of soft cohesive soil and fine grain with an N-value of 3, 4 or less. In this sampling, the holding sleeve 2 (sampling sleeve) is smoothly inserted into the ground to a target depth to extract samples.

A stainless steel thin pipe or a brass thin pipe having a length of 1.0 m and an inner diameter of 75 mm may be used as the holding sleeve 2 (sampling sleeve).

For a slightly stiff cohesive soil having an N-value of approximately 4 to 20, there may be used the Denison (double pipe) sampling in which an outer pipe of a thin-walled pipe is rotated and pushed into the ground while a peripheral soil is washed.

For a sandy soil or rudaceous soil, the triple pipe sampling may be carried out.

For a rudaceous soil or soft rock soil, the core pack sampling, by which a sample is put into a thin vinyl tube for extraction, may be used.

In an embodiment according to the present invention, in particular, by use of a vinyl tube as the holding sleeve 2 to hold a sample in the vinyl tube for extraction, the liquefied sample can advantageously be seen by sight after slitting the vinyl tube recovered on the ground with a knife. However, another extraction method or apparatus may also be adopted.

### [3] Two Sorts of Vibration

There has already been well known and marketed an apparatus configured to cause the vibrator 5 to apply vertical vibration to the casing 1 so that the casing 1 along with the holding sleeve 2 are inserted into the ground, thereby holding a soil inside the holding sleeve 2 as a sample 3.

The vibration applied by the vibrator 5 to the casing 1 for the insertion of the casing 1 in the above is referred to as “insertion vibration.”

Other than “insertion vibration,” the apparatus of an embodiment according to the present invention is characterized by applying vibration acceleration corresponding to a seismic intensity described later as a “seismic wave vibration.”

In other words, the apparatus of an embodiment according to the present invention is characterized by applying two sorts of vibration, “insertion vibration” and “seismic wave vibration,” to the casing 1 and the sample 3.

### [4] Insertion Vibration

The insertion vibration is a vibration for inserting the casing 1 into the ground as described above, and a frequency thereof depends on a size of the vibrator 5. The frequency e.g. 47 Hz is adopted for a type ECO-1VIII-CF of YBM Co., Ltd.

There are other well-known apparatuses, such as those manufactured by TOHO CHIKAKOKI Co., Ltd., those manufactured by Tone Engineering Corporation.

### [5] Seismic Wave Vibration

Seismic wave vibration is a vibration acceleration to be applied to the casing 1 having been inserted to a predetermined depth e.g. 5 m into the ground, and applied to the sample 3 inside the casing 1.

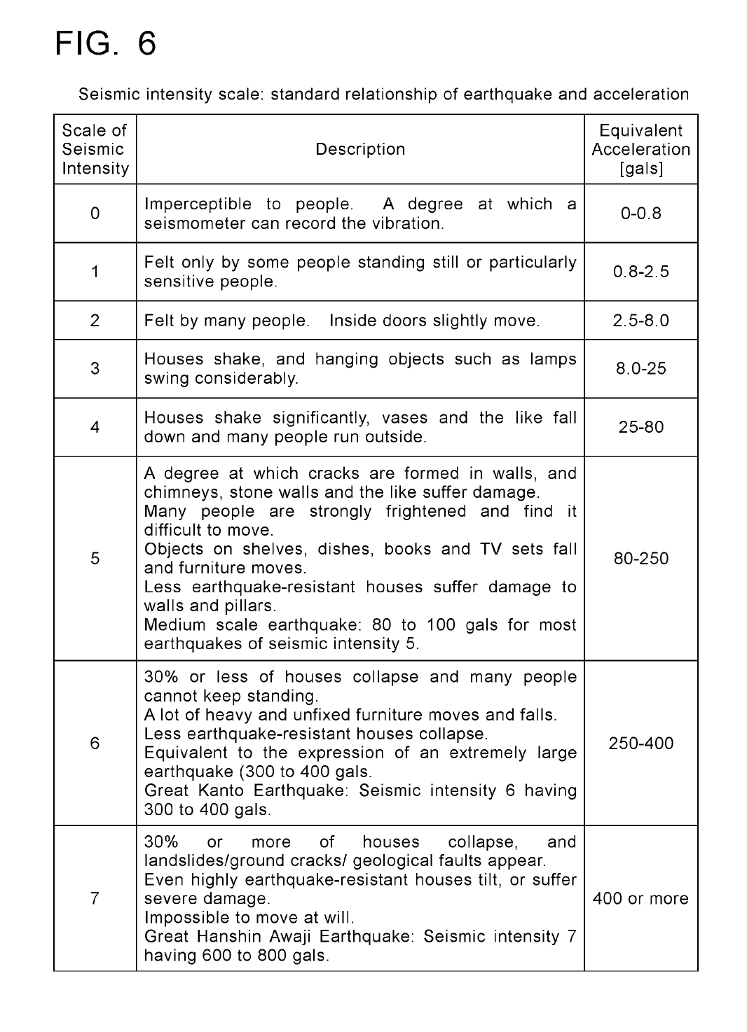

The vibration acceleration to be applied corresponds to a seismic intensity prepared in advance by the Meteorological Agency (FIG. 6).

As one example, the vibration acceleration corresponding to a seismic intensity 3 (at which intensity, houses shake, and hanging objects such as lamps swing considerably) is 8.0 to 25 gals.

As another example, the vibration acceleration corresponding to seismic intensity 7 (at which intensity, 30% or more of houses collapse) is determined to be 400 gals or more.

Liquefaction of the held soil is accelerated by mechanically applying the vibration acceleration corresponding to these gal values to the sample 3 inside the casing 1.

The vibration acceleration can be obtained by a=2πf·v=(2πf)²x showing that a predetermined vibration acceleration “a” can be obtained by adjusting a frequency “f” of the vibrator, a vibratory displacement “x,” a vibratory velocity “v,” and the like.

The direction of the vibratory displacement of vibration acceleration may be one of vertical vibration, horizontal vibration, or a combined vibration of both.

The seismic wave vibration may be provided by either the different vibrator from the vibrator configured to apply the insertion vibration for inserting the casing 1 or the same vibrator.

### [6] Other Structures for Insertion

In the structure of the example described above, the casing 1 is inserted by applying the insertion vibration. Other structures, which are configured such that the casing 1 is inserted by rotation without any vibration, may be used.

When the rotation is not enough in intensity to separate sticking, the casing 1 may be inserted by a combination of the rotation and the vibration.

### [7] Other Structures For Application Of Seismic Wave

In the structures of the above-described examples, the sample 3 inside the casing 1 is caused to vibrate by applying the vibration acceleration corresponding to a seismic intensity to the casing 1 itself. There may be used, however, other structures configured such that the vibration acceleration is applied directly to the sample 3 inside the casing 1 instead of the vibration acceleration being applied to the casing 1.

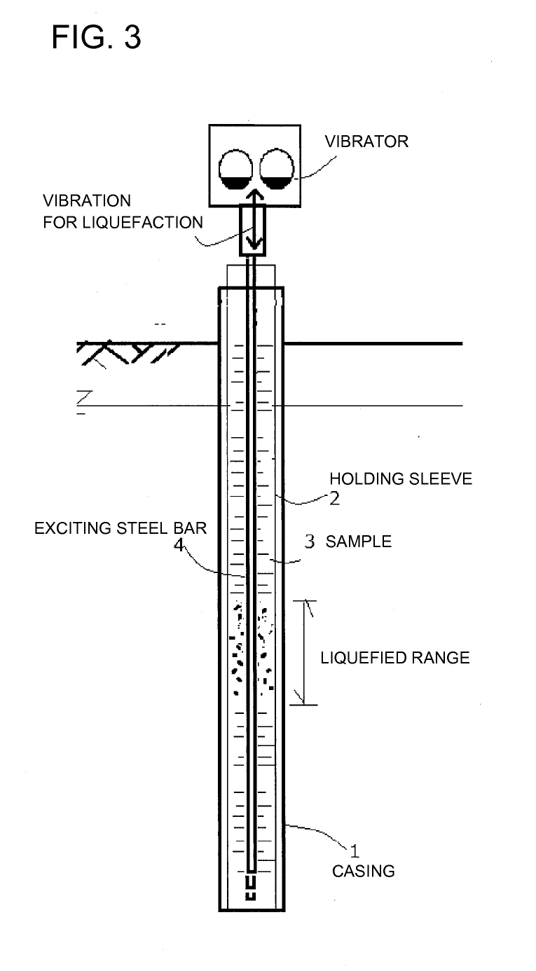

In such a case, a long exciting steel bar 4 or the like may be inserted into the sample 3, as shown in FIG. 3, for applying to the exciting steel bar 4 a vibration acceleration corresponding to a seismic intensity, thereby accelerating liquefaction of the sample 3, inside the casing 2, caused directly to vibrate.

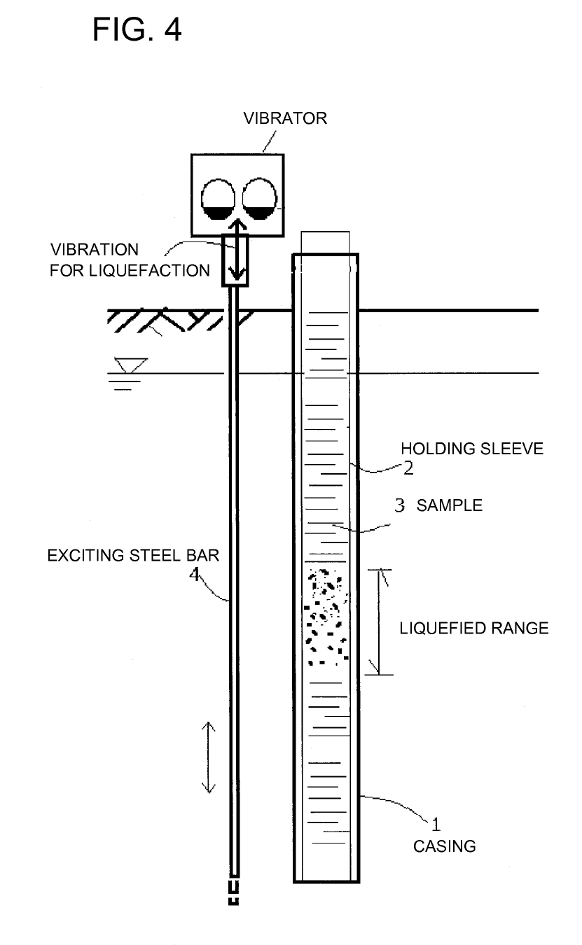

The above exciting steel bar 4 may also be inserted into the ground outside the casing 1, as shown in FIG. 4, for applying to the exciting steel bar 4 a vibration acceleration corresponding to a seismic intensity, thereby accelerating liquefaction of the sample 3, inside the casing 2, caused to vibrate by the ground outside the casing 2.

In either of the above structures, not only vertical vibration but also horizontal vibration or a combination of vertical/horizontal vibrations may be applied.

### [8] Sample Extraction Method

Subsequently, a method of extracting the sample 3 through the use of the core pack sampling in the above sample extraction apparatus will be described. It is to be noted, however, the sample extraction method is not limited to the core pack sampling.

### [9] Application of Insertion Vibration

A vinyl bag or the like is installed inside the casing 1 as the holding sleeve 2.

A tip of the holding sleeve 2 and a tip of the casing 1 are opened.

Then, the casing 1 is inserted into the target ground for investigation.

In such a case, the casing 1 is inserted by applying vertical insertion vibration, rotation, or a combination of rotation and vibration.

As the casing 1 is inserted, the soil of the ground i.e. the sample 3 as it is in a cut-out cylindrical shape enters an interior of the holding sleeve 2 installed inside the casing 1.

In order to assess liquefaction under various seismic conditions, a plurality of casings 1 corresponding to the number of the conditions are inserted into the target ground.

For example, in order to assess liquefaction under seismic intensity 3, seismic intensity 4, and seismic intensity 5, three casings 1 are inserted into the target ground at three points, respectively, for extracting the samples 3 at their respective points.

### [10] Application Of Seismic Wave Vibration

When the casing 1 is inserted to a predetermined depth, the insertion is stopped and the tip is closed, and vibration acceleration corresponding to a seismic intensity is applied to the sample 3 inside the casing 1 as seismic wave vibration so as to accelerate liquefaction.

Vibration acceleration corresponding to a seismic intensity at the timing may be applied, as described above, by any one of the methods of: application of vibration to the casing 1 itself; application of vibration to the sample 3 inside the casing 1; and application of vibration to the soil of the ground outside the casing 1.

In the soil including a layer having a risk of liquefaction due to seismic motion, only the corresponding range inside the holding sleeve 2 is liquefied.

In such a case, various vibration accelerations are applied to the samples 3, respectively, in such a manner that, for example: the vibration acceleration having 8.0 to 25 gals corresponding to seismic intensity 3 is applied to the first sample 3; the vibration acceleration having 25 to 80 gals corresponding to seismic intensity 4 is applied to the second sample 3; and the like.

### [11] Recovery Of Sample 3

Whether the sample 3 inside the holding sleeve 2 is liquefied or not cannot be assessed at ground intensity. For this reason, the holding sleeve 2 as it is along with the casing 2 is pulled up to the ground.

When the samples 3 at deep locations are extracted, a work of pulling up the sample 3 after having applied seismic wave vibration thereto is repetitively performed a plurality of times. In this case, the technique of recovering the sample 3 while preventing the sample from falling off has been well known.

Then, the holding sleeve 2 is pulled out from the casing 1, and is horizontally placed in a sample extraction box.

As described above, various vibration accelerations are applied to the samples 3 extracted at a plurality of points.

### [12] Assessment Of Liquefaction

After the recovery of the holding sleeve 2 up to the ground, the holding sleeve 2 is disassembled.

The range of liquefaction is in a squashy state clearly different from that of other sound layer ranges.

Therefore, the depth and range of liquefaction can be checked by sight or touch without expert knowledge about soil properties.

When the holding sleeve 2 is a vinyl bag, the sample 3 can be pulled out by slitting the bag open.

When the holding sleeve 2 is a thin-walled steel pipe, the sample 3 can be pulled out by pushing the sample 3 inside the pipe.

Therefore, a result, which shows e.g. the target ground liquefied under seismic intensity 4 while not liquefied under seismic intensity 3, can be recognized through the use of the actual soil of a plurality of samples 3 recovered in the above manner, without the need for hypothetical conditions or calculations.

### REFERENCE NUMERALS

1 Casing
2 Holding sleeve
3 Sample
4 Exciting steel bar
5 Vibrator

What is claimed is:

1. A sample extraction apparatus for extracting samples from a target ground in order to assess whether liquefaction occurs in the target ground, the apparatus comprising:
a casing;
a holding sleeve, installed inside the casing, for holding the samples; and
a vibrator configured to apply vibration accelerations corresponding to different seismic intensities to the samples in the casing, respectively, after the casing is inserted into the target ground to hold the samples of the target ground in the holding sleeve, so that a different liquefaction condition is provided for each sample, whereby
said apparatus is configured such that, after having applied different vibration accelerations to the samples in the casing inserted into the target ground, respectively, said apparatus can resultantly extract the samples so that a liquefaction assessment can be made under a plurality of seismic conditions.

2. The sample extraction apparatus according to claim 1, wherein the vibration accelerations applied to the samples are generated by causing the casing to vibrate.

3. The sample extraction apparatus according to claim 1, wherein the vibration accelerations applied to the samples are generated by causing the samples to vibrate inside the casing.

4. The sample extraction apparatus according to claim 1, wherein the vibration accelerations applied to the samples are generated by causing the ground to vibrate outside the casing.

5. A sample extraction method for the purpose of assessing as to whether liquefaction occurs in the target ground, the method comprising the steps of:
inserting the casing into the target ground to hold the samples of the target ground in the holding sleeve, by using the apparatus according to claim 1;
thereafter, applying vibration accelerations corresponding to different seismic intensities to the samples in the casing so as to provide the samples with different liquefaction conditions, respectively, by using the apparatus according to claim 1; and
thereafter, retrieving aboveground each sample, extracted by using the apparatus according to claim 1, for making a liquefaction assessment.
